(19) United States
(12) Patent Application Publication
(10) Pub. No.: US 2014/0046241 A1
(43) Pub. Date: Feb. 13, 2014

(54) METHOD FOR TREATING NON-ACNEIC OILY SKIN

(71) Applicants: Corinne Granger, Paris (FR); Helene Pernot, Paris (FR)

(72) Inventors: Corinne Granger, Paris (FR); Helene Pernot, Paris (FR)

(73) Assignee: L'OREAL, Paris (FR)

(21) Appl. No.: 13/992,950

(22) PCT Filed: Dec. 12, 2011
(86) PCT No.: PCT/IB2011/055611

§ 371 Date: Oct. 28, 2013

Related U.S. Application Data
(60) Provisional application No. 61/427,237, filed on Dec. 27, 2010.

(30) Foreign Application Priority Data
Dec. 13, 2010 (FR) 1060447

(51) Int. Cl. A61N 5/06; A61M 35/00

(52) U.S. Cl. CPC A61N 5/0616; A61M 35/00
USPC 604/20; 607/88; 607/89; 607/90

(57) ABSTRACT

The present invention relates to a method for cosmetic treatment of non-acneic oily skin, comprising the step consisting in: exposing said non-acneic oily skin to a first quasi-monochromatic light of artificial origin having a dominant wavelength peak between 300 and 700 nm, better still between 400 and 650 nm, even better still between 560 and 620 nm.

The present invention relates to the cosmetic, i.e. non-therapeutic, treatment of non-acneic oily skin.

The skin is rich in sebaceous glands and is continually renewed. The secretion of sebum is a normal phenomenon which is useful to both the skin and the head of hair. Sebum is normally an agent for moisturizing the epidermis. It is the natural product of the sebaceous gland, which is an annex of the pilosebaceous unit. It is essentially a more or less complex mixture of lipids. Sebum protects the skin and also the scalp and gives the hair sheen by lubricating the cuticle.

Unfortunately, a hypersecretion of sebum, or seborrhoea, may lead to esthetic disorders. Thus, an excessive secretion of sebum may result in oily skin with a shiny or glistening appearance and it may also promote the appearance of an oily dandruff condition of the scalp or oily dandruff. It may be accompanied by an increase in pore size. For example, stress, fatigue and the winter period may be factors that intensify these conditions in the majority of people. Among the population having oily skin, subjects can be found who have endocrine disorders or neurological disorders, or obese subjects. It is also possible to find adolescents, people suffering from excess hormones (in particular male hormones), menstruating women or menopausal women who have oily skin.

There is therefore a need to overcome these problems by providing a method for treating oily skin.

Methods for treating acne, which aim to reduce the proliferation of P. Acnes, are known from patent applications US 2006/0212025 and US 2006/0200213.

Presented in U.S. Pat. No. 6,183,773 and U.S. Pat. No. 6,600,951 are methods for treating disorders of the sebaceous gland by means of light emitted by a laser.

Application US 2009/0299268 has the objective of treating acne. The treatment makes it possible to reduce the secretion of sebum and gives rise to the eradication of the bacteria.

Application GB 2 356 570 teaches treating acne by the emission of light at three different wavelengths, namely within the following ranges: 365-465 nm, 585-645 nm and 646-710 nm.

Finally, application US 2005/0055070 relates to the treatment of acne by light in order to destroy the bacteria, owing to the stimulation of the production of free radicals via a photochemical reaction.

Acne is the main one of the most common forms of dermatosis. It is most common at the age of puberty. It is linked to the proliferation of certain local germs such as Propionibacterium acnes (P. Acnes). Acneic and acne-prone individuals usually have oily, oily-prone or combination skin.

The invention aims to treat non-acneic oily skin by providing a method for the cosmetic (non-therapeutic) treatment of non-acneic oily skin, comprising the steps consisting in:
exposing the non-acneic oily skin to a first quasi-monochromatic light of artificial origin having a dominant wavelength peak between 300 and 700 nm, better still between 400 and 650 nm, even better still between 560 and 620 nm, in particular of the order of around 590 nm.

The expression “monochromatic light” is understood to mean light which consists only of a single wavelength. According to the invention of this method, the expression “quasi-monochromatic light” is understood to mean light emitting a spectrum of wavelengths having a dominant peak at one wavelength. According to the invention, this spectrum has a spectral width at mid-height of at most ±50 nm and a spectral width at the base of at most ±100 nm. The spectral width at mid-height is defined as the width of the spectrum at half of the power of the dominant peak. The spectral width at the base is defined as the width of the spectrum at 10% of the power of the dominant peak.

The applicant has observed that it was possible to thus treat skin which has not developed a clinical manifestation of acne in order to make the skin less oily, less glistening and less shiny.

The term “skin” denotes the skin of the face, of the body and the scalp. The skin treated may or may not be wrinkled.

The expression “non-acneic skin” denotes healthy skin free of the clinical manifestations of acne, such as the presence of numerous acne pimples. In other words, over the whole region of skin exposed to the light with the method according to the invention, the skin is not clinically acneic. The entire region of skin exposed to the light is free of a region that has developed acne. The expression “clinical manifestations of acne” is understood to mean the presence on the skin of acne lesions.

Hyperseborrhoeic oily skin is characterized by an exaggerated secretion and excretion of sebum. The expression “oily skin” denotes skin which obtains a score of greater than 95 μg/cm² on the sebumeter. The expression “very oily skin” denotes a score on the sebumeter of greater than 120 μg/cm² and the expression “excessively oily skin” denotes a score on the sebumeter of greater than 140 μg/cm².

Such skin is also often associated with a lack of desquamation, a glistening complexion, a thick skin texture, enlarged pores or an irregular relief, which manifestations are perceived as being skin imperfections or esthetic defects. The appearance and/or visibility of the pores is also a characteristic of oily skin. The shininess of the skin is also linked to the enlargement of the pores. Oily skin is also characterized by glistening skin, sometimes of oily appearance, which is thick, having enlarged pilosebaceous pores.

Sebumeter

The sebumeter makes it possible to measure the production of sebum over time. The amount of sebum excreted at the surface of the skin is evaluated using a Sebumeter® SM180 (Courage & Khazaka).

This is a photometric method. A tape of synthetic material, which becomes transparent on contact with the absorbed lipids, is applied to the measurement region for precisely 30 seconds.

Its transparency then increases proportionally with the amount of sebum of the hydrolipid film with which it is in contact.

A recording by reflectometry makes it possible to quantify the increase in transmitted light and thus to determine the total mass of lipids excreted per unit of surface area (in μg·cm⁻²).

A measurement on the forehead after careful defatting with 70° alcohol is performed.

30 minutes later, a new measurement is taken. The amount of sebum excreted per unit of surface area and per unit of time may thus be calculated.

The amount of sebum may also be evaluated using a Sebutape®.

Sebutape®

The Sebutape® makes it possible to measure the amount of sebum produced over a given period. Use is made of a Sebutape® with the reference S100 from the company CuDerm Corp. Tx, USA, also available from the company Monaderm. The Sebutape® is applied and gently pressed on the skin at the temples after careful defatting with 70° alcohol, and left in place in contact with the skin for a time of 30 minutes. Next, the Sebutape® is removed and then brought into contact with a transparent plastic film. The score is determined visually using an appropriate scale of 0 to 5.

Typically, oily skin according to the invention has a score of greater than 2.

Dermascore

The Dermascore makes it possible to visualize certain characteristics of the skin as a function of the polarization of light, namely the pores of the skin with the polarized light parallel and the colour of the skin and also the heterogeneities of the skin with the polarized light perpendicular. Images are captured and an analysis carried out by comparison with the images from an atlas.

The present invention reduces the secretion of sebum. Oily skin is also often associated with a lack of desquamation, a glistening complexion, a thick skin texture and an increased pore size, which manifestations are perceived as being esthetic disorders which the treatment according to the invention also aims to rectify.

The treatment according to the invention advantageously makes it possible to prevent and/or treat the glistening appearance of the skin. For the purposes of the present invention, the term “prevent” is understood to mean at least partly reducing the risk of manifestation of a given phenomenon. Partial reduction implies that the risk remains but to a lesser degree than before the implementation of the invention.

The treatment in accordance with the invention advantageously makes it possible to prevent and/or treat skin of blotchy, dull and/or uneven, waxy or yellowish appearance, or even of morbid appearance.

The treatment in accordance with the invention advantageously makes it possible to prevent and/or treat the esthetic disorders associated with an oily scalp, such as a hypersecretion of sebum, or seborrhoea, which may promote the appearance of an oily dandruff condition of the scalp or oily dandruff.

The treatment according to the invention may thus prove to be very particularly effective:
for preventing and/or treating oily skin,
for improving the comfort of oily skin and scalps,
for treating and/or preventing and/or avoiding esthetic disorders of the scalp associated with excessive excretions and/or secretions of sebum,
for preventing and/or treating oily scalps, and in particular the oily dandruff conditions of the scalp,
for re-establishing a balanced ecoflora of the oily scalp.

One hypothesis put forward for explaining the effect of the treatment, without being bound by this explanation, is that the treatment according to the invention acts on the sebaceous gland, decreasing its activity.

The method may have an effect on the physiological and clinical signs of oily skin, in particular the quantity of sebum and the sebum quality. Another effect of the treatment method according to the invention may be to reduce the size of the pores. Yet another effect may be to reduce the visibility of acne scars.

The treatment method according to the invention does not aim to destroy the P. Acnes bacterium. The method makes it possible to reduce the production of sebum by at least 8% and to significantly reduce, by at least 7%, the P. Acnes by a modification of the nutritional conditions for the growth of P. Acnes. The method according to the invention may make it possible to limit the growth of P. Acnes. The method according to the invention may make it possible to reduce the population of P. Acnes, considering the reduction in the presence of oil on the skin, which tends to promote the proliferation of the bacterium. The reduction in the amount of P. Acnes present on the skin may lead to the reduction in the risk of the occurrence of small acne pimples and also a reduction in sebum production in so far as the presence of P. Acnes in excess tends to promote sebum production.

The method according to the invention is more particularly intended for treating individuals who are between 20 and 60 years old, better still between 25 and 50 years old, when their skin does not suffer from acne.

The method according to the invention may be carried out by exposing the face, by region or in its entirety, the only scalp, by region or in its entirety, or the body or a part of the body having an oily skin problem to the light source.

The method according to the invention may be carried out by exposing the face, by region or in its entirety, the only scalp, by region or in its entirety, or the body or a part of the body to the light source with a static apparatus that may or may not be in contact with the region treated.

The expression “static apparatus” is understood to mean that an apparatus does not have to be moved with respect to the face or the head.

As a variant, the apparatus may be used successively over several regions in order to cover a larger treatment area. Over each region, the apparatus is kept immobile.

The method according to the invention may be carried out with a support that emits light according to the invention, in contact with the skin of the face, of the scalp or of the body. The support may be a material that emits light. The support may be at least partially, or even completely, made of a textile. The method according to the invention may be carried out by a touch-control apparatus, a rigid or flexible screen, a mirror, a housing or a glass that emits light according to the invention.

The treatment does not cause thermal lesions, the light power being weak.

The spectrum of the light emitted may comprise a first quasi-monochromatic light corresponding to the first light above, alone or in combination with one or more other quasi-monochromatic lights.

Besides the exposure to the first quasi-monochromatic light as defined above, it is possible to expose the non-acneic oily skin to a second quasi-monochromatic light of artificial origin having a dominant wavelength peak between 700 and 1000 nm, better still between 800 and 900 nm, for example of the order of 870 nm. This second light is a red or infrared light. The exposure to the red or infrared light may be carried out simultaneously or successively to the exposure to the first light mentioned above.

The source producing the red or infrared light may be the same as or different from the source producing the first light mentioned above. It is possible to carry out the treatment using a single light source configured to emit two different quasi-monochromatic lights. As a variant, it is possible to carry out the treatment using two different sources that emit two different quasi-monochromatic lights. The two sources may be activated simultaneously or successively.

It is also possible to expose the skin to a third quasi-monochromatic blue light, having a dominant wavelength peak between, for example, 400 and 450 nm, in particular of the order of 410 to 420 nm. This third additional light may be emitted by a light source different from the light source(s) emitting the first light and optionally the second red or infrared light.

The first light may be dominant with respect to the other light(s). The dominant wavelength peak of the first light may have an intensity greater than the other dominant wavelength peak(s) of the other lights. The first light may, for example, represent more than 50%, better still more than 60% and even better still more than 70% of the total energy of all the light received.

The light source(s) generating the light to which the skin is exposed may comprise at least one of the following: an LED, an LED matrix, an OLED, a laser, an incandescent lamp equipped with a dichroic filter, this list not being limiting.

The use of an OLED may be preferred in so far as they make it possible to apply the light as close as possible to the skin. They may be integrated into a mask or a patch.

The light source(s) may comprise at least one quasi-monochromatic LED. The expression “quasi-monochromatic LED” denotes an LED having an emission spectrum that comprises a dominant wavelength peak with a spectral width at mid-height of at most +50 nm and a spectral width at the base of the peak of at most ±100 nm. As a variant, the light source may comprise at least one bichromatic LED, that is to say the emission spectrum of which comprises several quasi-monochromatic lights, for example two quasi-monochromatic lights, for example one quasi-monochromatic light for which the dominant peak is between 560 nm and 620 nm, of greater intensity, and one quasi-monochromatic light for which the dominant peak is between 700 nm and 1000 nm, of lower intensity.

The light source(s) may be other than a laser.

The light, in particular the first light having a wavelength between 300 and 700 nm as defined above, may be pulsed. The optional second and/or third lights may also be pulsed, for example with pulses of the same duration and interpulse intervals of the same duration as the first light. As a variant, the pulsation characteristics of these three lights may differ from one another. In another variant, at least one of the second and third lights may be continuous. The pulses may have a duration of between 100 and 500 ms, better still between 200 and 300 ms, for example of the order of 250 ms. The light may be pulsed with interpulse intervals having a duration of between 50 and 200 ms, better still between 70 and 150 ms, for example of the order of 100 ms.

As a variant, all the light emitted may be continuous.

The surface power density of the light received by the non-acneic oily skin during a treatment may be less than 40 mW/cm², preferably between 1 and 20 mW/cm², better still between 1 and 10 mW/cm², for example of the order of 5 mW/cm².

The surface energy density received by the non-acneic oily skin during the treatment, over one day, may be less than 4 J/cm², better still between 20 mJ/cm² and 1 J/cm², for example of the order of 175 mJ/cm².

It is possible to expose a same region of non-acneic oily skin to the light in accordance with the invention for a duration of less than 20 minutes, for example between 20 and 100 seconds, for example of the order of 35 seconds or of 70 seconds.

In one example of the implementation of the invention, when the skin is positioned at a distance between 0 and 10 cm from the light source, the surface power density received is less than 40 mW/cm² and the surface energy density received less than 4 J/cm².

It is possible to carry out said treatment at least once a day, at least one day per week, better still two days per week, or as a variant five days per week, or even every day, for a period of at least two weeks.

In one example of the implementation of the invention, said treatment is carried out at least once a week for a duration of between two and twelve weeks.

The skin thus treated may be oily, very oily, or even excessively oily, as defined above.

The treatment may be carried out on the skin of a subject after evaluating the skin of this subject, the evaluation having made it possible to determine the “oily” nature of the subject's skin. The evaluation may result from a measurement carried out on the subject's skin, for example using a Sebumeter™ or a Sebutape™, or else by self-evaluation. The self-evaluation may, for example, result from responses given by the subject to a detailed questionnaire regarding sensations of oily skin or of glistening skin, or visual observations of the state of the skin under various observation conditions, for example at certain moments of the day or a certain time after washing.

The treatment may be carried out with a light having a power of the dominant wavelength in the range 700 nm-1000 nm that is less than at least one quarter of the power of the dominant wavelength in the range 560 nm-620 nm.

Another subject of the invention is, independently or in combination with the aforegoing, a method for the cosmetic treatment of non-acneic oily skin, comprising the step consisting in:
exposing the non-acneic skin to a light source having a dominant wavelength between 300 and 700 nm, better still between 400 and 650 nm, better still between 560 and 620 nm, preferably of the order of 590 nm so as to reduce the production of sebum by 8% at least and so as to reduce the P. Acnes by at least 7% by a modification of the nutritional conditions for the growth of P. Acnes.

The treatment according to the invention may be carried out without application of product. More specifically, product cannot be applied to the region to be treated, in particular in the hour before exposure to the light. Product cannot be applied to the treated region, in particular in the hour after exposure to the light.

Pretreatment

As a variant, the treatment may be carried out with application of product. The method may thus also comprise the following step:
applying a cosmetic product to the skin, prior to the exposure to the light.

Post-Treatment

As a variant or additionally, the treatment may be followed by an application of product. The method may thus also comprise the following step:
applying a cosmetic product to the skin, after the exposure to the light.

Compositions

The pretreatment or post-treatment products may be such as described below.

It may be, for example, a cleansing product such as Cetaphil (water, cetyl alcohol, propylene glycol, sodium lauryl sulfate, stearyl alcohol, methylparaben, propylparaben, butylparaben). The product applied may be relatively mild, making it possible to only gently cleanse the skin, for example to remove make-up, but not being a product that is too degreasing.

As a variant or additionally, it may be a moisturizing product. The moisturizing product may, for example, have one of the following general compositions A or B:

Composition A: Cream in the Form of a Water-in-Oil Emulsion

| Chemical name | Concentration |
|---|---|
| WATER | QS 100% |
| GLYCEROL | 7 |
| METHYL p-HYDROXYBENZOATE | 0.3 |
| MAGNESIUM SULFATE | 0.7 |
| OXYETHYLENATED | 3.75 |
| POLY(METHYLCETYL)(DIMETHYL)- | |
| METHYLSILOXANE | |
| (20/75/5—VISCOSITY: 3000 cSt) | |
| POLYGLYCERYL-4 ISOSTEARATE | 1.25 |
| ISOHEXADECANE | 7.75 |
| ISONONYL ISONONANOATE | 7.75 |
| PROPYL p-HYDROXYBENZOATE | 0.25 |
| MIXTURE OF ACETYLATED ETHYLENE GLYCOL | 0.5 |
| STEARATE AND GLYCERYL TRISTEARATE | |
| POLY(STEARYL ACRYLATE) | 1.3 |
| VITAMIN E ACETATE | 0.2 |
| IODOPROPYNYL BUTYLCARBAMATE | 0.2 |
| 5-(n-OCTANOYL)SALICYLIC ACID | 0.3 |
| CYCLOHEXASILOXANE | 8.7 |
| ACRYLATES COPOLYMER | 0.5 |
| FRAGRANCE | 0.2 |
| AMORPHOUS SILICA MICROSPHERES | 3 |
| GLYCOLIC ACID | 0.1 |
| ALGAE EXTRACT | 0.5 |
| ETHYLENEDIAMINETETRAACETIC ACID, | 0.05 |
| TETRASODIUM SALT | |
| COPPER PIDOLATE | 0.05 |
| ZINC PIDOLATE | 0.25 |
| NYLON-12 (and) CAPRYLOYL SALICYLIC ACID | 2.7 |
| (and) TOCOPHEROL | |
| TOTAL | 100 |

Composition B: Cream in the Form of an Oil-in-Water Emulsion

| Chemical name | Concentration |
|---|---|
| WATER | QS 100% |
| GLYCEROL | 5 |
| EDTA | 0.2 |
| CONDENSATE OF ETHYLENE OXIDE AND OF | 0.5 |
| PROPYLENE OXIDE AND OF ETHYLENE OXIDE | |
| (MW: 14000) (128 EO/54 PO/128 EO) | |
| NYLON-12 PARTICLES FILLED WITH TOTAROL, | 0.5 |
| 5-(n-OCTANOYL)SALICYLIC ACID AND | |
| β-GLYCYRRHETINIC ACID | |
| PURE SODIUM HYDROXIDE | 0.52 |
| PHENYL TRIMETHYLSILOXY TRISILOXANE | 2 |
| (VISCOSITY: 20 cSt - MW: 372) | |
| ALKYL METHACRYLATE-GRAFT-POLY | 0.3 |
| DIMETHYLSILOXANE COPOLYMER | |
| ACRYLIC ACID/STEARYL METHACRYLATE | 0.9 |
| COPOLYMER POLYMERIZED IN AN ETHYL | |
| ACETATE/CYCLOHEXANE MIXTURE | |
| 0.3 mm LONG 0.9 dtex POLYAMIDE FIBERS | 2 |
| WASHED AT HIGH TEMPERATURE THEN | |
| DABBED DRY | |
| LACTATE OF BRANCHED C12-13 ALCOHOLS | 2 |
| 5-(n-OCTANOYL)SALICYLIC ACID | 0.15 |
| FRAGRANCE | 0.25 |
| ACRYLAMIDE/SODIUM 2- | 0.5 |
| ACRYLAMIDOMETHYLPROPANESULFONATE | |
| COPOLYMER AS AN INVERSE EMULSION AT | |
| 40% IN ISOPARAFFIN/WATER | |
| TITANIUM OXIDE-MICA-TIN OXIDE (58/41/1) | 0.5 |
| ZINC PIDOLATE | 0.25 |
| ETHYLENEDIAMINETETRAACETIC ACID, | 0.05 |
| TETRASODIUM SALT | |
| EPERUA FALCATA BARK EXTRACT | 0.1 |
| ETHYL ALCOHOL | 9 |
| SALICYLIC ACID POWDER | 0.5 |
| POLYETHYLENE BEADS (SIZE: 8-10 microns) | 3 |
| POLYETHYLENE POWDER | 5 |
| DYE | QS |
| TOTAL | 100 |

It is also possible to apply a topical cream, comprising an active agent. The activity of the active agent may be intensified owing to the exposure to light.

It is also possible to apply a matting product, that is to say a product having an immediate effect for solving the problem of oily skin, the exposure to light making it possible to have a more long-term effect.

It is also possible to carry out a care routine, comprising the compositions A and B applied successively. Composition A is for example applied in the evening and composition B in the morning.

A method according to the invention may comprise the topical application of a composition to the non-acneic oily skin, the skin of the face and/or the scalp.

The composition may comprise, in a physiologically acceptable medium, an active agent for caring for oily skin, for example a desquamating agent, for example 5-(n-octanoyl)salicylic acid.

The composition may comprise, in a physiologically acceptable medium, one or more agents from the following list: matting agent, exfoliant or abrasive filler, sebo-regulating agent, desquamating agent, calmative, anti-irritant, anti-inflammatory, antioxidant, cicatrizing agent, astringent.

The term “matting agent” means agents intended to make the skin visibly more matt and less shiny.

The matting effect of the agent and/or composition containing it may especially be evaluated using a gonioreflectometer, by measuring the ratio R between the specular reflection and the diffuse reflection. A value of R of less than or equal to 2 generally indicates a matting effect.

The matting agent may especially be chosen from a rice starch or a corn starch, kaolinite, silicas, talc, a pumpkin seed extract, cellulose microbeads, plant fibers, synthetic fibers, in particular polyamide fibers, expanded acrylic copolymer microspheres, polyamide powders, silica powders, polytetrafluoroethylene powders, silicone resin powders, acrylic copolymer powders, wax powders, polyethylene powders, powders of elastomeric crosslinked organopolysiloxane coated with silicone resin, talc/titanium dioxide/alumina/silica composite powders, amorphous mixed silicate powders, acrylic polymer powders, silicate particles and especially mixed silicate particles, and mixtures thereof.

Examples of matting agents that may especially be mentioned include:
rice or corn starch, in particular an aluminum starch octenyl succinate sold under the name Dry Flo® by the company National Starch;
kaolinite;
silicas;
talc;
a pumpkin seed extract as sold under the name Curbilene® by the company Indena;
cellulose microbeads as described in patent application EP 1 562 562;
fibers, such as silk fibers, cotton fibers, wool fibers, flax fibers, cellulose fibers extracted especially from wood, from vegetables or from algae, polyamide (Nylon®) fibers, modified cellulose fibers, poly-p-phenylene terephthamide fibers, acrylic fibers, polyolefin fibers, glass fibers, silica fibers, aramid fibers, carbon fibers, Teflon® fibers, insoluble collagen fibers, polyester fibers, polyvinyl chloride or polyvinylidene chloride fibers, polyvinyl alcohol fibers, polyacrylonitrile fibers, chitosan fibers, polyurethane fibers, polyethylene phthalate fibers, fibers formed from a mixture of polymers, resorbable synthetic fibers, and the mixtures thereof described in patent application EP 1 151 742;
expanded acrylic copolymer microspheres such as those sold by the company Expancel under the name Expancel 551®;
fillers with an optical effect as described in patent application FR 2 869 796, in particular:
   polyamide (Nylon®) powders, for instance Nylon 12 particles of the Orgasol type from Atofina, with a mean size of 10 microns and a refractive index of 1.54,
   silica powders, for instance Silica beads SB150 from Miyoshi with a mean size of 5 microns and a refractive index of 1.45,
   polytetrafluoroethylene powders, for instance PTFE Ceridust 9205F from Clariant, with a mean size of 8 microns and a refractive index of 1.36,
   silicone resin powders, for instance the silicone resin Tospearl 145A from GE Silicone with a mean size of 4.5 microns and a refractive index of 1.41,
   acrylic copolymer powders, especially of polymethyl (meth)acrylate, for instance the PMMA particles Jurymer MBI from Nihon Junyoki, with a mean size of 8 microns and a refractive index of 1.49, or the Micropearl M100® and F 80 ED® particles from the company Matsumoto Yushi-Seiyaku,
   wax powders, for instance the paraffin wax particles Microease 114S from Micropowders, with a mean size of 7 microns and a refractive index of 1.54,
   polyethylene powders, especially comprising at least one ethylene/acrylic acid copolymer, and in particular consisting of ethylene/acrylic acid copolymers, for instance the particles Flobeads EA 209 from Sumitomo (with a mean size of 10 microns and a refractive index of 1.48),
   elastomeric crosslinked organopolysiloxane powders coated with silicone resin, especially with silsesquioxane resin, as described, for example, in U.S. Pat. No. 5,538,793. Such elastomer powders are sold under the names KSP-100, KSP-101, KSP-102, KSP-103, KSP-104 and KSP-105 by the company Shin-Etsu, and
   talc/titanium dioxide/alumina/silica composite powders such as those sold under the name Coverleaf AR-80 by the company Catalyst & Chemicals,

and mixtures thereof;
compounds that absorb and/or adsorb sebum as described in the same patent application FR 2 869 796. Mention may be made especially of:
   silica powders, for instance the porous silica microspheres sold under the name Silica Beads SB-700 sold by the company Miyoshi, the products Sunsphere® H51, Sunsphere® H33 and Sunsphere® H53 sold by the company Asahi Glass; the polydimethylsiloxane-coated amorphous silica microspheres sold under the name SA Sunsphere® H-33 and SA Sunsphere® H-53 sold by the company Asahi Glass;
   amorphous mixed silicate powders, especially of aluminum and magnesium, for instance the product sold under the name Neusilin UFL2 by the company Sumitomo;
   polyamide (Nylon®) powders, for instance Orgasol® 4000 sold by the company Atochem; and
   acrylic polymer powders, especially of polymethyl methacrylate, for instance Covabead® LH85 sold by the company Wackherr; of polymethyl methacrylate/ethylene glycol dimethacrylate, for instance Dow Corning 5640 Microsponge® Skin Oil Adsorber sold by the company Dow Corning, or Ganzpearl® GMP-0820 sold by the company Ganz Chemical; of polyallyl methacrylate/ethylene glycol dimethacrylate, for instance Poly-Pore® L200 or Poly-Pore® E200 sold by the company Amcol; of ethylene glycol dimethacrylate/lauryl methacrylate copolymer, for instance Polytrap® 6603 sold by the company Dow Corning;
   silicate particles, such as alumina silicate;
   mixed silicate particles, such as:
      magnesium aluminum silicate particles, such as saponite or hydrated magnesium aluminum silicate with a sodium sulfate sold under the trade name Sumecton® by the company Kunimine;
      the magnesium silicate, hydroxyethylcellulose, black cumin oil, marrow oil and phospholipids complex or Matipure® from Lucas Meyer;

and mixtures thereof.

Preferred matting agents that may be used according to the invention include a pumpkin seed extract, a rice or corn starch, kaolinite, silicas, talc, polyamide powders, polyethylene powders, acrylic copolymer powders, expanded acrylic copolymer microspheres, silicone resin microbeads and mixed silicate particles, and mixtures thereof.

Abrasive Fillers or Exfoliants

As exfoliants that may be used in compositions according to the invention, examples that may be mentioned include exfoliating or scrubbing particles of mineral, plant or organic origin. Thus, use may be made, for example, of polyethylene beads or powder, nylon powder, polyvinyl chloride powder, pumice, ground materials derived from apricot kernels or walnut shells, sawdust, glass beads, alumina and mixtures thereof.

Mention may also be made of Exfogreen® from Solabia (bamboo extract), extracts of strawberry achenes (strawberry achenes from Greentech), peach kernel powder or apricot kernel powder and, finally, mention is made, in the field of plant powders with an abrasive effect, of cranberry seed powder.

Mention will be made, as preferred abrasive fillers or exfoliants according to the invention, of peach kernel powder, apricot kernel powder, cranberry seed powder, extracts of strawberry achenes or bamboo extracts.

Sebo-Regulating or Anti-Seborrhoeic Agents

The term “sebo-regulating or anti-seborrhoeic agents” especially means agents capable of regulating the activity of the sebaceous glands.

Mention may be made especially of:
retinoic acid, benzoyl peroxide, sulfur, vitamin B6 (or pyridoxine), selenium chloride and sea fennel;
mixtures of extract of cinnamon, of tea and of octanoyl glycine such as Sepicontrol A5 TEA from SEPPIC;
the mixture of capryloyl glycine, sarcosine and Cinnamomum zeylanicum extract sold especially by the company SEPPIC under the trade name Sepicontrol A5®;
zinc salts such as zinc gluconate, zinc pyrrolidone carboxylate (or zinc pidolate), zinc lactate, zinc aspartate, zinc carboxylate, zinc salicylate and zinc cysteate;
copper salts, in particular copper pidolate;
extracts of plants of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all sold, for example, by the company Maruzen;
meadowsweet (Spiraea ulmaria) extracts, such as the product sold under the name Sebonormine® by the company Silab;
extracts of the alga Laminaria saccharina, such as the product sold under the name Phlorogine® by the company Biotechmarine;
mixtures of extracts of salad burnet root (Sanguisorba officinalis/Poterium officinale), of ginger rhizomes (Zingiber officinalis) and of cinnamon bark (Cinnamomum cassia), such as the product sold under the name Sebustop® by the company Solabia;
linseed extracts, such as the product sold under the name Linumine® by the company Lucas Meyer;
Phellodendron extracts, such as those sold under the name Phellodendron extract BG by the company Maruzen or Oubaku liquid B by the company Ichimaru Pharcos;
mixtures of argan oil, of Serenoa serrulata (saw palmetto) extract and of sesame seed extract, such as the product sold under the name Regu SEB® by the company Pentapharm;
mixtures of extracts of willowherb, of Terminalia chebulas, of nasturtium and of bioavailable zinc (microalgae), such as the product sold under the name Seborilys® by the company Greentech;
extracts of Pygeum africanum, such as the product sold under the name Pygeum africanum sterolic lipid extract by the company Euromed;
extracts of Serenoa serrulata, such as the products sold under the name Viapure Sabal by the company Actives International or those sold by the company Euromed;
mixtures of extracts of plantain, of Berberis aquifolium and of sodium salicylate, such as the product sold under the name Seboclear® by the company Rahn;
clove extract, such as the product sold under the name Clove extract powder by the company Maruzen;
argan oil, such as the product sold under the name Lipofructyl® by Laboratoires Sérobiologiques;
lactic protein filtrates, such as the product sold under the name Normaseb® by the company Sederma;
extracts of the alga Laminaria, such as the product sold under the name Laminarghane® by the company Biotechmarine;
sugar cane extracts, such as the product sold under the name Policosanol® by the company Sabinsa;
oligosaccharides of the alga Laminaria digitata, such as the product sold under the name Phycosaccharide AC by the company Codif;
sulfonated shale oil, such as the product sold under the name Ichthyol Pale by the company Ichthyol;
meadowsweet (Spiraea ulmaria) extracts, such as the product sold under the name Cytobiol Ulmaire by the company Libiol;
sebacic acid, especially sold in the form of a sodium polyacrylate gel under the name Sebosoft by the company Sederma;
glucomannans extracted from konjac tuber and modified with alkylsulfonate chains, such as the product sold under the name Biopol Beta by the company Arch Chemical;
extracts of Sophora angustifolia, such as those sold under the name Sophora powder or Sophora extract by the company Bioland;
extracts of Cinchona succirubra bark, such as the product sold under the name Red Bark HS by the company Alban Muller;
extracts of Quillaja saponaria, such as the product sold under the name Panama wood HS by the company Alban Muller;
glycine grafted onto an undecylenic chain, such as the product sold under the name Lipacide UG OR by the company SEPPIC;
the mixture of oleanolic acid and of nordihydroguaiaretic acid, such as the product sold in the form of a gel under the name AC.Net by the company Sederma;
phthalimidoperoxyhexanoic acid;
tri(C12-C13)alkyl citrate sold under the name Cosmacol® ECI by the company Sasol; tri(C14-C15)alkyl citrate sold under the name Cosmacol® ECL by the company Sasol;
10-hydroxydecanoic acid, and especially mixtures of 10-hydroxydecanoic acid, of sebacic acid and of 1,10-decanediol, such as the product sold under the name Acnacidol® BG by the company Vincience;
specific PPAR-γ activators such as those described in application WO 2005/053632;
extracts of plants of the genus Silybum;
sapogenins or plant extracts containing them, in particular extracts of Dioscoreas rich in diosgenin; and
extracts of Eugenia caryophyllata containing eugenol and eugenyl glucoside;
and mixtures thereof.

As preferred sebo-regulating agents that can be used according to the invention, mention will be made of:
sea fennel;
mixtures of extract of cinnamon, of tea and of octanoyl glycine such as Sepicontrol A5 TEA from SEPPIC;
the mixture of capryloyl glycine, sarcosine and Cinnamomum zeylanicum extract sold especially by the company SEPPIC under the trade name Sepicontrol A5®;
zinc salts such as zinc gluconate, zinc pyrrolidone carboxylate (or zinc pidolate), zinc lactate, zinc aspartate, zinc carboxylate, zinc salicylate and zinc cysteate;
copper salts, in particular copper pidolate;
meadowsweet (Spiraea ulmaria) extracts, such as the product sold under the name Sebonormine® by the company Silab;
extracts of the alga Laminaria saccharina, such as the product sold under the name Phlorogine® by the company Biotechmarine;
mixtures of extracts of salad burnet root (Sanguisorba officinalis/Poterium officinale), of ginger rhizomes (Zingiber officinalis) and of cinnamon bark (Cinnamomum cassia), such as the product sold under the name Sebustop® by the company Solabia;
sapogenins or plant extracts containing them, in particular extracts of Dioscoreas rich in diosgenin;
and mixtures thereof.

Antiperspirants may also be mentioned, such as: aluminum and/or zirconium salts; complexes of zirconium hydroxychloride and of aluminum hydroxychloride with an amino acid, such as those described in U.S. Pat. No. 3,792,068, commonly known as “ZAG complexes”. Such complexes are generally known under the name ZAG (when the amino acid is glycine). ZAG complexes ordinarily have an Al/Zr ratio ranging from about 1.67 to 12.5 and a metal/Cl ratio ranging from about 0.73 to 1.93. Among these products, mention may be made of aluminum zirconium octachlorohydrex GLY, aluminum zirconium pentachlorohydrex GLY, aluminum zirconium tetrachlorohydrate GLY and aluminum zirconium trichlorohydrate GLY.

Among the aluminum salts that may be mentioned are aluminum chlorohydrate, aluminum chlorohydrex, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, alum salts, aluminum sulfate, aluminum zirconium octachlorohydrate, aluminum zirconium pentachlorohydrate, aluminum zirconium tetrachlorohydrate, aluminum zirconium trichlorohydrate and more particularly the aluminum chlorohydrate sold by the company Reheis under the name Microdry Aluminum Chlorohydrate or by the company Guilini Chemie under the name Aloxicoll PF 40. Aluminum zirconium salts are for example the product sold by the company Reheis under the name Reach AZP-908-SUF, “activated” aluminum salts, for example the product sold by the company Reheis under the name Reach 103 or by the company Westwood under the name Westchlor 200.

Among the other deodorant active agents, mention may also be made of zinc salts such as zinc salicylate, zinc sulfate, zinc chloride, zinc lactate and zinc phenolsulfonate; chlorhexidine and the salts; diglycerol monocaprate, diglycerol monolaurate, glycerol monolaurate; and polyhexamethylene biguanide salts.

Antimicrobial Agents

The expression “antimicrobial agents” is understood to mean agents that have effects on the specific flora of oily skin, such as for example P. acnes.

These effects may be either bactericidal, or effects that act against bacterial adhesion (that prevent and/or reduce the adhesion of microorganisms), or effects that act on the biofilm of bacteria so as to prevent multiplication thereof.

Mention may especially be made of the active agents and preserving agents with antimicrobial activity mentioned in application DE 103 24 567, which is incorporated into the present invention by reference.

Mention may also be made of: a hop cone extract (HOP CO2-TO extract from Flavex), a St. John's Wort extract (St. John's Wort CO2-TO extract from Flavex), asiatic acid, extracts of roots of Scutellaria baicalensis, as in BMB—CF from Naturogin, piroctone olamine, citrollic acid, sperillic acid, ethylhexylglycerin (Sensiva from Schülke), glyceryl caprylate/caprate (Capmul from ABITEC), calcium sodium phosphosilicate, such as Bioactive Glass Powder from Schott, Actysse Premier BG from Schott, silicon oxides from Ciba, Metashines (silver derivatives), extracts of bearberry, such as Gatuline Equalizing from Gattefossé, 10-hydroxy-2-decanoic acid, such as Acnacidol P from Vincience, sodium ursolate, azelaic acid, diiodomethyl-p-tolyl sulfone or Amical Flowable from Angus, malachite from Maprecos, Zincare from Elementis GmbH, Arlatone Dioic from Unichema; phthalimidoperoxyhexanoic acid or Eureco HC from Chemron Corporation; ellagic acid; 2,4,4′-trichloro-2′-hydroxydiphenyl ether (or triclosan), 1-(3′,4′-dichlorophenyl)-3-(4′-chlorophenyl)urea (or triclocarban), 3,4,4′-trichlorocarbanilide, 3′,4′,5′-trichlorosalicylanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopirox olamine, undecylenic acid and its salts, benzoyl peroxide, 3-hydroxybenzoic acid, 4-hydroxybenzoic acid, phytic acid, N-acetyl-L-cysteine, lipoic acid, azelaic acid and its salts, arachidonic acid, resorcinol, 3,4,4′-trichlorocarbanilide, octopirox or piroctone olamine, octoxyglycerin or octoglycerin, octanoyl glycine (Lipacid C8G® from SEPPIC), caprylyl glycol, 10-hydroxy-2-decanoic acid, dichlorophenyl imidazole dioxolane and its derivatives described in patent application WO 93/18743, zinc derivatives and in particular zinc pidolate (Zincidone® from Solabia), copper derivatives and in particular copper pidolate (Cuivridone® from Solabia), salicylic acid and its derivatives, iodopropynyl butyl carbamate, 3,7,11-trimethyldodeca-2,5,10-trienol or farnesol, phytosphingosines; Sepicontrol® from SEPPIC, an argania tree extract, such as Argapure LS9710®, Sebosoft® from Sederma, quaternary ammonium salts, such as cetyltrimethylammonium salts or cetylpyridinium salts, ethanol, etc. and mixtures thereof.

Mention may in particular be made, as agents which prevent and/or reduce the adhesion of microorganisms, of: phytanetriol and derivatives thereof as described in patent application EP 1 529 523, plant oils such as wheatgerm oil, calendula oil, castor oil, olive oil, avocado oil, sweet almond oil, groundnut oil, jojoba oil, sesame seed oil, apricot kernel oil, sunflower oil and macadamia oil, described in patent EP 1 133 979, or else other fatty substances, such as disodium cocoamphodiacetate, oxyethylenated (7 EO) glyceryl cocoate, the Poloxamers, hexadecenyl succinate 18, octoxyglyceryl palmitate, octoxyglyceryl behenate, dioctyl adipate, PPG-15 stearyl ether or the tartrate of branched C12-C13 dialcohols which are described in patent EP 1 129 694.

Mention may be made, in particular with regard to the propagation of P. acnes, of pentylene glycol, nylon-6,6 (fibers of polyamide-6,6), rice bran oil, Celvol 540 PV alcohol (polyvinyl alcohol 72962), Akorex L from Karlshamns or fructose derivatives.

Mention may also be made of certain surfactants having an antimicrobial effect, such as sodium cocoamphoacetate or disodium cocoamphodiacetate, such as Miranol C2M Conc. NP, betaines, such as the cocoyl betaine Genagen KB from Clariant, sodium lauryl ether sulfate, such as Emal 270 D from Kao, decyl glucoside, such as Plantacare 2000 UP, malates of branched C12-C13 dialcohols, such as Cosmacol EMI, propylene glycol monoesters, such as propylene glycol monolaurate, monocaprylate or monocaprate, sodium lauroyl oat amino acid, such as Proteol OAT, lauryl dimethylamine betaine, such as Empigen BB/LS, and also polyquaternary ammoniums, such as Quaternium-24 or Bardac 2050 from Lonza and those described in the L′Oreal patent FR 0 108 283.

Use will be made in the compositions of the invention, as preferred antimicrobial agents, of an agent chosen from caprylyl glycol, zinc derivatives, including zinc pidolate (Zincidone® from Solabia), copper derivatives, including copper pidolate (Cuivridone® from Solabia), octoglycerin or octoxyglycerin, 10-hydroxy-2-decanoic acid and mixtures thereof.

Desquamating Agents

The term “desquamating agent” means any compound capable of acting:
either directly on desquamation by promoting exfoliation, such as β-hydroxy acids, in particular salicylic acid and its derivatives (including 5-(n-octanoyl)salicylic acid); α-hydroxy acids, such as glycolic acid, citric acid, lactic acid, tartaric acid, malic acid or mandelic acid; urea; gentisic acid and its derivatives; oligofucoses; cinnamic acid; Saphora japonica extract; resveratrol and certain jasmonic acid derivatives;
or on the enzymes involved in the desquamation or degradation of corneodesmosomes, glycosidases, stratum corneum chymotryptic enzyme (SCCE) or even other proteases (trypsin, chymotrypsin-like). Mention may be made of aminosulfonic compounds and in particular N-(2-hydroxyethyl)piperazine-N-2-ethanesulfonic acid (HEPES); 2-oxothiazolidine-4-carboxylic acid (procysteine) derivatives; derivatives of α-amino acids of glycine type (as described in EP-0 852 949, and also sodium methyl glycine diacetate sold by BASF under the trade name Trilon M); honey; sugar derivatives such as O-octanoyl-6-D-maltose and N-acetylglucosamine.

Mention may be made, as other desquamating agents which can be used in the composition according to the invention, of oligofructoses, EDTA and its derivatives, Laminaria extracts, O-linoleyl-6-D-glucose, (3-hydroxy-2-pentylcyclopentyl)acetic acid, glyceryl trilactate, O-octanyl-6′-D-maltose, S-(carboxymethyl)cysteine, silicon-comprising salicylate derivatives, as in patent EP 0 796 861, oligofucases, as in patent EP 0 218 200, salts of 5-acylsalicylic acid, active agents having effects on transglutaminase, as in patent EP 0 899 330, and jasmonic acid and derivatives, as in patent applications EP 1 333 022 and EP 1 333 021.

Exfolactive® from Silab (extract of Opuntia ficus-indica flower) or Soypon O® from Kawaken Fine Chemicals (sodium cocoyl sarcosinate).

Mention may be made, as preferred desquamating agents, of β-hydroxy acids, such as 5-(n-octanoyl)salicylic acid; urea; glycolic acid, citric acid, lactic acid, tartaric acid, malic acid or mandelic acid; N-(2-hydroxyethyl)piperazine-N-2-ethanesulfonic acid (HEPES); Saphora japonica extract; honey; N-acetylglucosamine; sodium methyl glycine diacetate; and mixtures thereof.

More preferably still, use will be made, in the compositions of the invention, of a desquamating agent chosen from 5-(n-octanoyl)salicylic acid; urea; N-(2-hydroxyethyppiperazine-N-2-ethanesulfonic acid (HEPES); Saphora japonica extract; honey; N-acetylglucosamine; sodium methyl glycine diacetate; and mixtures thereof.

Calmatives or Anti-Irritants

Mention may in particular be made of the calmatives or anti-irritants mentioned in applications WO 2004/105736 and DE 10324567, incorporated in the present invention by way of reference.

Mention may be made, as specific calmatives which can be used in the composition according to the invention, of: procyanidolic oligomers, vitamins E, C, B5 or B3, dextran sulfate, caffeine and its derivatives, pentacyclic triterpenes and the plant extracts containing them, β-glycyrrhetinic acid and its salts or derivatives (stearyl glycyrrhetate, 3-stearoyloxyglycyrrhetic acid or glycyrrhetinic acid monoglucuronide) and also the plants containing them (e.g.: Glycyrrhiza glabra), oleanolic acid and its salts, ursolic acid and its salts, boswellic acid and its salts, betulinic acid and its salts, a Paeonia suffruticosa and/or lactiflora extract, zinc salicylate, phycosaccharides from Codif, a Laminaria saccharina extract, Centella asiatica extracts, canola oil, bisabolol, the phosphoric diester of vitamin E and C, such as Sepivital EPC® from SEPPIC, camomile extracts, allantoin, omega-3 unsaturated oils, such as musk rose oil, blackcurrant oil, Echium oil or fish oil, calophyllum oil, plankton extracts, capryloyl glycine, Seppicalm VG® (Nymphaea alba and sodium palmitoyl proline) from SEPPIC, a Pygeum extract, a Boswellia serrata extract, a Centipeda cunninghamii extract, a Helianthus annuus extract, in particular Helioxine from Silab, a Linum usitatissimum extract, such as Sensiline from Silab, tocotrienols, Cola nitida extracts, piperonal, a clove extract, an Epilobium angustifolium extract, aloe vera, a Bacopa monnieri extract, phytosterols, cornflower water, rose water, dextran, as in Modulene® from Vincience, a mint extract, in particular an extract of mint leaves, such as Calmiskin® from Silab, anise derivatives, filamentous bacteria, such as Vitreoscilla filiformis, such as described in patent EP 761 204, a rose extract, such as Herbasol Rose Extract, Stimutex AS from Pentapharm, salts of alkaline earth metals, in particular strontium, niacinamide, and mixtures thereof.

Use will be made, as preferred calmatives, of an agent chosen from a rose extract, a clove extract, dextran, as in Modulene® from Vincience, a mint extract, such as Calmiskin® from Silab, a mixture of a Nymphaea alba extract and sodium palmitoyl proline, such as Seppicalm VG® from SEPPIC, anise derivatives, a Paeonia suffruticosa and/or lactiflora extract, and mixtures thereof.

Anti-inflammatory agents

Mention may in particular be made of the anti-inflammatory agents mentioned in applications WO 2004/105736 and DE 10324567, incorporated in the present invention by way of reference.

Mention may be made, as specific anti-inflammatory agents which can be used according to the invention, of cortisone, hydrocortisone, indomethacin, betamethasone, azelaic acid, acetaminophen, diclofenac, clobetasol propionate and mixtures thereof.

Mention will be made, as preferred anti-inflammatory agent, of azelaic acid.

Antioxidants

This expression is understood to mean agents having an antioxidant activity (which prevent the oxidation of squalene and the formation of comedones).

Mention may in particular be made of tocopherol and its esters, in particular tocopheryl acetate, BHT and BHA.

Mention may also be made of polyphenols, tannic acid, epigallocatechins and the natural extracts containing them, anthocyans, rosemary extracts, olive leaf extracts, green tea, resveratrol and its derivatives, Pycnogenol, ergothioneine, N-acetylcysteine, biotin, chelating agents, idebenone, plant extracts, such as Pronalen Bioprotect™ from Provital, antiradicals, such as vitamin E, coenzyme Q10, bioflavonoids, SODs, phytantriol, lignans, melatonin, pidolates or glutathione.

Cicatrizing Agents

Examples of cicatrizing agents that may especially be mentioned include:

allantoin, urea, wheatgerm oil, certain amino acids, such as hydroxyproline, arginine or serine, and also Madonna lily extracts (e.g.: Phytélène Lys 37EG 16295 from Indena), a yeast extract, such as the cicatrizing agent LS 7225B from LS (Cognis), tamanu oil, Saccharomyces cerevisiae extract or Biodynes TRF from Arch Chemical, oat extracts, chitosan and derivatives, carrot extracts, Artemia extract or GP4G from Vincience, sodium acexamate, lavandin extracts, honey or propolis extracts, ximeninic acid and its salts, such as acido ximeninico from Indena, Rosa rugosa oil, Souci Ami Liposolible from Alban Muller, horsetail extracts, Lemon Herbasol from Cosmetochem, Helichrysum extracts, β-glucan and derivatives, shea butter and its purified fractions, modified exopolysaccharides and alkylsulfonated polyaminosaccharides.

As preferred cicatrizing agents according to the invention, use will be made of tamanu oil, sodium acexamate, honey extracts, horsetail extracts and Helichrysum extracts, and mixtures thereof.

Astringents

According to the invention, the term “astringents” means agents for combating the dilation of the sebaceous follicles.

Mention may be made, as astringents which can be used in the composition according to the invention, of Laricyl LS8865® from Cognis, Phytofirm LS9120® from Cognis, Tanlex VE/VB® from Ichimaru Pharcos, laponite, aluminum salts, Centella extracts (e.g., Plantactiv Centella from Cognis), Varisoft 432 CG® from Degussa, horse chestnut extracts, mallow extracts from Hammamelis, Almondermin LS 3380® from Cognis, burdock extracts, Extrapone 9 Special® from Symrise, skullcap extracts, meadowsweet extracts (e.g., Cytobiol Ulmaire from Libiol), Herb Extract B1348® from Bell Flavors & Fragrances, acacia, elm, white willow, cinnamon, birch or meadowsweet extracts, Panama sapogenins, zinc phenolsulfonate from Interchemical, gentian, cucumber or walnut extracts, or the Epilami mixture from Alban Muller.

As preferred astringents according to the invention, use will be made of skullcap extracts, meadowsweet extracts, meadowsweet extracts, gentian extracts and burdock extracts, and mixtures thereof.

### Tests

Tests were carried out using a GentleWaves™ LED device from Light Bioscience emitting at a dominant wavelength of around 595 nm (visible yellow light) and also at a second wavelength of 870 nm, at a lower power, with a total surface power density of 5 mW/cm², the light being pulsed with a cycle of 250 ms of emissions and 100 ms between the emissions. The power of the wavelength at 870 nm represents at most 10% to 15% of the power of the dominant wavelength at 595 nm.

Test 1

Test carried out on 94 women with oily skin: 3 groups are treated over one half of the face only randomly with the invention:
Group 1: 35 s twice a week for four weeks (8 sessions), the subject applies her customary cosmetic cream over the whole of the face, morning and evening.
Group 2: 35 s twice a week for four weeks followed by 35 s once a week for four weeks (12 sessions), the subject applies her customary cosmetic cream over the whole of the face, morning and evening.

Group 3: 35 s twice a week for four weeks followed by 35 s once a week for four weeks (12 sessions), with application of compositions A and B, compared to the use of the customary cream alone on the other half of the face not treated by the invention.

Results of the Sebutape®

The Sebutape values after 2, 4 or 8 weeks of treatment were compared to the Sebutape values before treatment.

In group 1, the mean of the differences is statistically significant and equal to −0.3062 from 2 weeks of treatment by the invention, whereas this difference is not statistically significant on the side not treated by the invention.

In group 3, the mean of the differences is statistically significant and equal to −0.373 from 4 weeks and this difference remains statistically significant at 8 weeks on the side treated by the invention and the compositions A and B.

Results of the Sebumeter: The Sebumeter values after 2, 4 or 8 weeks of treatment were compared to the Sebumeter values before treatment.

In group 2, the mean of the differences is statistically significant and equal to −19.7813 from 2 weeks of treatment. This difference remains (=−16.6563) statistically significant at 4 weeks of treatment by the invention, whereas this difference is not statistically significant on the untreated side.

In group 3, the mean of the differences is statistically significant and equal to −24.800 from 4 weeks of treatment. This difference remains (=−23.967) statistically significant at 8 weeks of treatment by the invention plus the compositions A and B, whereas this difference is not statistically significant on the side not treated by the invention.

Results of the Dermascore: The Dermascore values after 2, 4 or 8 weeks of treatment were compared to the Dermascore values before treatment by the invention.

In group 2, the mean of the differences is statistically significant and equal to −0.1438 from 2 weeks of treatment. This difference is not statistically significant on the side not treated by the invention.

In group 3, the mean of the differences is statistically significant and equal to −0.227 from 4 weeks of treatment by the invention plus the compositions A and B, whereas this difference is not statistically significant on the side not treated at 4 weeks of treatment by the invention without the compositions A and B.

The effectiveness of the treatment was very clearly detected by the volunteers. The skin appears less oily (97% G3 from 2 weeks), smoother (97% G1 and G3 at 8 weeks), healthier (97% G3 at 8 weeks), the skin texture appears finer (97% G3 at 4 weeks), the pores more tightened (93% G3) and less visible (93% G3 from 2 weeks).

The comparison of the three treatment methods regarding the percentage satisfaction of the women with respect to the criterion “pore visibility” at 2 and at 4 weeks shows a significant difference in favor of the group of women using the treatment with the care routine comprising the compositions A and B (group 3) relative to those who have the same method of use of the device but not in combination with the compositions A and B (group 2).

All the treatment methods specified above made it possible to significantly reduce the sebaceous excretions measured by means of the Sebutape™ from 4 weeks in comparison with the untreated side. The combination of the treatment by the device with a specific product makes it possible to achieve a significant reduction of this same parameter after 2 weeks.

Test 2

Test carried out on 100 (60/40) women and men treated over the whole of the face randomly by the invention according to the groups: a group A of subjects is treated for 1×35 s, twice a week, over four weeks. Another group B of subjects is treated for 2×35 s consecutively, twice a week, over four weeks. Another group C of subjects is treated for 1×35 s, twice a day, morning and evening, twice a week, over four weeks. Another group D of subjects is treated for 1×35 s, every day, five times a week from Monday to Friday, over four weeks. Another group E of subjects is treated for 2×35 s consecutively, every day, five times a week from Monday to Friday, over four weeks.

Results Relating to the Pore Size:

The study on the pore size was carried out by analysis and evaluation on the basis of photographs of the subjects taken at the end of the treatment and compared to photographs taken before the treatment by the invention. A significant difference was demonstrated in the appearance of the pores for the subjects treated in group D (7.6%), group C (6.5%) and in group B (6.5%). No difference was revealed regarding the appearance of the pores in groups A and E.

Test 3

Test carried out on 124 subjects with oily skin treated by the invention over half of the face (left or right) randomly for 70 seconds, once a day, 5 days in a row, over 4 weeks.

A statistically significant reduction (p<0.001) in the pore size was observed over the half of the face treated for 1 month by the invention compared to the initial state before treatment.

At 1 month of treatment, a significant difference equal to 0.16 (p<0.05) of the variations of sebum excretion is observed compared to the base state between the side treated by the invention and the side not treated by the invention.

An evaluation carried out by a blind expert with respect to the half of the face treated or not treated over 1 month by the invention showed the following results: on the half of the face treated over 1 month by the invention, the expert evaluated a less oily and less shiny skin in 61% and 62% of the subjects respectively, whereas on the side not treated by the invention at 1 month the expert evaluated a less oily and less shiny skin in 38% and 42% of the subjects respectively. These differences are statistically significant. The effect of the treatment according to the subject was evaluated by self-questionnaires after 1 month of treatment by the invention: the subjects considered that their skin was less oily on the half of the face treated (66%) and 50% for the half of the face not treated by the invention. Regarding the pore size, the subjects considered that the pores were less visible (63%) and tightened (66%) on the half of the face treated and 48% and 47% respectively for the half of the face not treated.

The invention will be better understood on reading the following detailed description of exemplary embodiments of the invention and on examining the appended drawing, in which:

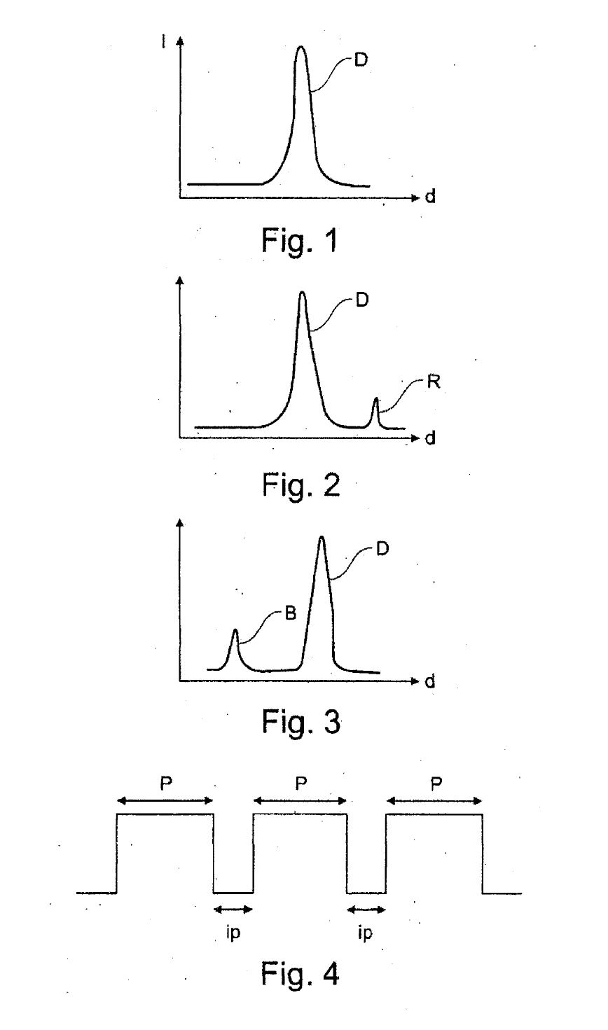

FIGS. 1 to 3 illustrate light spectra (Intensity I) as a function of the wavelength (λ) emitted in accordance with the invention, and

FIG. 4 illustrates a sequence of pulses in accordance with one implementation example of the invention.

### DEVICE

The method according to the invention is, for example, carried out by means of a device for applying light to the skin, comprising at least one source of quasi-monochromatic light of artificial origin making it possible to emit the first wavelength D, as illustrated in FIG. 1, and one or more exit window(s) for the light emitted.

The device may also be configured to emit a second quasi-monochromatic light of artificial origin having a wavelength peak R between 700 and 1000 nm, better still between 800 and 900 nm, for example of the order of around 870 nm, as illustrated in FIG. 2. This light is a red or infrared light. The source of red or infrared light may be the same as or different from that behind the first light mentioned above. The device may comprise a single light source configured to emit two different quasi-monochromatic lights. As a variant, the device may comprise two different sources that each emit at least one different quasi-monochromatic light, it being possible for the two sources to be activated simultaneously or successively.

The device may also be configured to emit a third additional quasi-monochromatic light of artificial origin, having a peak B of wavelength, for example, between 400 and 450 nm, in particular of the order of 410 to 420 nm, as illustrated in FIG. 3. This light is a blue light. The device may comprise an additional light source different from the light source(s) emitting the first light D and the red or infrared light R.

The device may be configured to emit a pulsed light with pulses having a duration p of between 100 and 500 ms, better still between 200 and 300 ms, for example of the order of 250 ms, and interpulse intervals ip having a duration of between 50 and 200 ms, better still between 70 and 150 ms, for example of the order of 100 ms, as illustrated in FIG. 4. As a variant, the device may be configured to emit a continuous light.

The surface power density of the light emitted may be less than 40 mW/cm², preferably between 1 and 20 mW/cm², better still between 1 and 10 mW/cm².

The surface energy density emitted may be less than 4 J/cm², better still between 20 mJ/cm² and 1 J/cm², for example of the order of 175 mJ/cm².

The device may be configured to emit light over a certain predefined operating period, then to switch off automatically. The operating period may be less than 20 minutes, for example between 20 and 100 seconds, for example of the order of 35 seconds or 70 seconds in certain exemplary embodiments.

The device may be configured so that the power of the dominant peak of the light R is at least less than a quarter of the power of the dominant peak of the light D.

The device may comprise means for conducting the light emitted by the source close to the skin, for example one or more optical fibers. The device may comprise a bundle of optical fibers conducting the light emitted by the source to a plurality of regions of the skin to be treated.

The device may be configured to make it possible to treat all of the subject's skin to be treated in a static manner. It may, for example, take the form of a helmet intended to cover the head, in order to enable treatment of the oily scalp. It may, as a variant, comprise several panels, each panel bearing one or more light sources, intended to be positioned in front of the face and at the sides of the face of a subject.

The device may comprise reliefs intended to be placed in contact with the skin of the subject when the device is placed on the subject in order to carry out the treatment, these reliefs making it possible to guarantee an adequate distance between the skin to be treated and the light source(s).

As a variant, the device comprises two curved LED panels that pivot relative to one another, a height-adjustable arm, a control unit and a power supply system. Each panel may consist of a matrix of at least 1000 LEDs.

As a variant, the device comprises a single curved LED panel connected to a control unit and to a power supply system. Each panel may consist of a matrix of at least 800 LEDs.

The invention is not limited to the exemplary embodiments that have just been described.

The expression “comprising a” should be understood as being synonymous with “comprising at least one”.

1. A method for cosmetically treating non-acneic oily skin, the method comprising:
exposing the non-acneic oily skin of a subject in need thereof to a first quasi-monochromatic light of artificial origin having a dominant wavelength peak between 300 and 700 nm.

2. The method of claim 1, wherein a production of sebum is reduced by 8% at least and a population of P. Acnes is reduced by at least 7% by a modification of the modifying at least one nutritional condition for P. Acnes growth.

3. The method of claim 2, further comprising:
exposing the non-acneic oily skin of a subject in need thereof, to a second quasi-monochromatic light of artificial origin having a dominant wavelength peak between 700 and 1000 nm.

4. The method of claim 1, wherein the first quasi-monochromatic light is pulsed with a pulse having a duration of between 100 and 500 ms.

5. The method of claim 1, wherein the first quasi-monochromatic light is pulsed with an interpulse interval having a duration of between 50 and 200 ms.

6. The method of claim 1, wherein the first quasi-monochromatic light received by the non-acneic oily skin has a surface power density of less than or equal to 40 mW/cm².

7. The method of claim 1, wherein the non-acneic oily skin is exposed to the first quasi-monochromatic light for a duration of less than 20 minutes.

8. The method of claim 1, wherein the cosmetically treating is carried out at least once a day and at least one day per week.

9. The method of claim 1, wherein the cosmetically treating is carried out at least once a week and for a duration of between two and twelve weeks.

10. The method of claim 1, the method further comprising:
exposing the non-acneic oily skin of the subject in need thereof to a third quasi-monochromatic blue light.

11. The method of claim 1, wherein a light source of the first quasi-monochromatic light comprises at least one selected from the group consisting of an LED, an LED matrix, an OLED, a laser, and an incandescent lamp equipped with a dichroic filter.

12. The method of claim 11, wherein the light source comprises at least one quasi-monochromatic LED.

13. The method of claim 1, further comprising:
applying a cosmetic product to the non-acneic oily skin of a subject in need thereof, prior to the exposing.

14. The method of claim 1, further comprising:
applying a cosmetic product to the non-acneic oily skin of a subject in need thereof, after the exposing.

15. The method of claim 1, comprising a topical application to the non-acneic oily skin of a composition comprising 5-(n-octanoyl)salicylic acid.

16. The method of claim 3, wherein the second quasi-monochromatic light is pulsed with a pulse having a duration of between 100 and 500 ms.

17. The method of claim 3, wherein the second quasi-monochromatic light is pulsed with an interpulse interval having a duration of between 50 and 200 ms.

18. The method of claim 3, wherein the second quasi-monochromatic light received by the non-acneic oily skin has a surface power density of less than or equal to 40 mW/cm².

19. The method of claim 3, wherein the non-acneic oily skin is exposed to the second quasi-monochromatic light for a duration of less than 20 minutes.
